(19) United States
(12) Patent Application Publication
(10) Pub. No.: US 2007/0038152 A1
(43) Pub. Date: Feb. 15, 2007

(54) TACTILE BREAST IMAGER AND METHOD FOR USE

(76) Inventors: Armen P. Sarvazyan, Lambertville, NJ (US); Vladimir Egorov, Princeton, NJ (US); Jae S Son, Rancho Palos Verdes, CA (US)

Correspondence Address: BORIS LESCHINSKY, P.O. BOX 72, WALDWICK, NJ, 07463, US

(21) Appl. No.: 11/459,985

(22) Filed: Jul. 26, 2006

Related U.S. Application Data
(62) Division of application No. 10/866,395, filed on Jun. 12, 2004, now abandoned.
(60) Provisional application No. 60/477,740, filed on Jun. 12, 2003.

(51) Int. Cl. A61B 5/103

(52) U.S. Cl. 600/587

(57) ABSTRACT

A method and device for breast examination adapted for easy home use including a tactile imager probe equipped with a pressure sensor array and a motion tracking system for simultaneously recording pressure and positioning data during the examination of a breast or any other soft tissue. The method includes a step of starting the examination from a known point or an anatomical mark such as a nipple or a sternum; moving the probe towards the area of interest and oscillating it thereabout while manually or automatically recording pressure and positioning data. Subsequent data analysis identifies the presence of a lesion, calculates its location relative to the probe, estimates the location of the probe relative to the anatomical landmark and finally calculates the location of the lesion relative to that anatomical landmark. The method allows repeating examinations over time with great accuracy as they all start from the same anatomical landmark. The probe includes provisions for easy hand grip or attaching to fingers of a patient, a cable connector or a wireless transmitter for transmitting the data out to a computer or another data analysis device, as well as manual data entry means allowing the patient herself to enter the positioning data.

### CROSS-REFERENCE DATA

A priority date benefit is claimed herein from a U.S. Provisional Patent Application No. 60/477,740 filed by the same inventors on Jun. 12, 2003 and entitled “Tactile breast imager”, which is incorporated herein in its entirety by reference. This application is a divisional application of a co-pending U.S. patent application Ser. No. 10/866,395 filed Jun. 12, 2004 with the same title and incorporated herewith in its entirety by reference.

This invention was made with government support under SBIR Grants No. R43 CA91392 and No. R43/44 CA69175 awarded by the National Institutes of Health, National Cancer Institute. The government has certain rights in this invention.

### BACKGROUND OF THE INVENTION

This invention relates generally to imaging of biological objects and, more particularly, to a method and apparatus for mass breast screening and detecting early changes in mechanical properties of breast tissue that are indicative of breast cancer and other breast pathologies, and, even more specifically, to the utilization of a hand-held self-palpation device for detecting and locating lesions in breast tissue.

### DESCRIPTION OF THE PRIOR ART

Breast cancer is one of the largest classes of malignant disease in women and the second leading cause of death among women in the United States. Approximately 1 woman in every 10 will develop breast cancer in her lifetime.

It has been shown that screening for breast cancer can reduce breast cancer mortality. Among women aged 50 and older, studies have demonstrated a 20% to 40% reduction in breast cancer mortality for women screened by mammography and clinical breast examination. Among women between 40 to 49 years of age, the mortality rate is reduced by 13% to 23%. These results suggest that further methods of preemptive mass screening could potentially reduce the mortality in all age group of women.

Although techniques such as computerized tomography, mammography, ultrasounds, and magnetic resonance imaging have greatly improved tumor surveillance over the past decade, there still remains a need for a simple, compact, easy to use, inexpensive and at the same time reliable and sensitive diagnostic device that each woman could use periodically for breast self-examination at home instead of manual palpation. Early detection of breast cancer represents a compelling goal in oncology.

Periodic palpation of the breasts by a physician and mammography often detect stage I breast tumors (the cancer is no wider than 2 centimeters in diameter and has not spread outside the breast). These examinations should be reasonably frequent, particularly in older women, in order to detect tumors before they can metastasize. However the cost of frequent examinations, plus the accumulated radiation exposure from frequent mammograms tend to limit such frequency. In addition, mammography may miss small tumors, especially in the dense breasts of younger women. Further, pregnant women should avoid exposure to radiation. Thus, there is a continuing need for improved methods and apparatus for very early detection of very small breast lumps that could be malignant, while avoiding radiation exposure.

Manual breast self-examination is a simple, worthwhile, atraumatic and non-hazardous method that is practiced worldwide. It has been shown that more frequent manual examinations increase the likelihood of detecting breast cancer, reduce the delay in treatment, detect tumors at an earlier clinical stage and smaller tumor size, and improve survival rates. The primary criticism of manual examination is that women do not examine their breasts properly. Several authors have stated that only 10-12 percent of women performing manual examination have correctly applied breast cancer detection methodology. It is well recognized, however, that at least 80 percent of all breast cancers are detected by women themselves. Manual breast examination is a viable and successful method of cancer detection and it is important that all women perform this monthly examination in a uniform manner. There are many methods of teaching manual breast examination. Such methods include films, lectures, mass media, brochures and instruction from health professionals. But nevertheless most women have difficulty in detecting small lesions and differentiating between harmful and harmless lesions and they have little or no knowledge of the various types of lumps, which may occur in the breast. Therefore a simple tactile device for home use having the ability to detect and recognize the different types of lesion will allow women and health professionals to recognize the dangerous tumor before it will become lethal.

Manual breast examination does have certain limitations. Again, the challenge is to differentiate significant palpable findings from the nonsignificant ones that do not feel much different. In many patients, the findings are not conclusive and the breast examiner/physician has difficulty in interpreting what his fingers feel in the breast. The question that must be answered is “is the nodularity which she feels within the limits of the normal physiologic variation in breast structure or does it represent a dominant tumor due to inflammatory or neoplastic disease?” It is apparent that a measurable difference in resistance may exist between significant and nonsignificant findings. Unfortunately, the human fingertip may not be sensitive enough to measure the difference. In fact, it is believed that palpation is not able to detect tumors of less than about one centimeter in size.

In order to increase the sensitivity of palpation and allow data acquisition and analysis, a number of devices and methods to detect breast tumors have been developed. Frei et al., U.S. Pat. Nos. 4,144,877 and 4,250,894, describe an instruments for breast examination that use a plurality of spaced piezoelectric strips which are pressed into the body being examined by a pressure member which applies a given periodic or steady stress to the tissue beneath the strips. U.S. Pat. Nos. 6,468,231; 5,524,636 and 5,860,934 issued to Sarvazyan (one of the inventors of the present invention) disclose a number of devices including a pressure sensor array, a data acquisition circuit and a data processing means. These patents are incorporated herein in their entirety by reference. Detection of nodules is achieved by analyzing the dynamic and spatial features of the pressure pattern while the probe is pressed to the tissue under investigation. U.S. Pat. No. 5,833,634 issued to Laird et al. discloses a tissue examination device that includes a transducer element for generating a signal in response to a force imposed on the transducer element in accordance with the varying properties of the underlying tissue structure and circuitry for detecting a variation in the signal as an indication of a localized area of stiffer tissue within the tissue.

A number of breast examination devices for clinical use based on computerized mechanical palpation have also been described. Mentioned above U.S. Pat. No. 5,860,934 discloses the device including an electronically controlled mechanical scanning unit incorporated into a patient support bed. The mechanical scanning unit includes a compression mechanism and positioning system, a local pressure source located opposite a pressure sensor array, and electronic control and interface circuitry. U.S. Pat. No. 6,091,981 issued to Cundari at al. describes a device that includes sensors producing signals in response to pressure imposed on the sensors as the sensors are pressed against the breast tissue. A location or a map of detected underlying tissue structure relative to a reference point is generated and displayed. U.S. Pat. No. 6,190,334 discloses an apparatus for automated breast palpation including an actuator having an extendable probe for contacting the tissue and an electronic control module. A signal processor receives the force and the displacement distance determinations from the electronic control module and analyzes these data to provide a visual data analysis indicating any lesion within the tissue. U.S. Pat. No. 6,192,143 describes a computer controlled apparatus for detecting breast tumors by mechanically palpating in a full surface scan manner in order to detect small lumps or other anomalies.

There have been multiple attempts to develop hand-held self-palpation devices for sensing regions of hardening in breast tissue and thus mimicking manual palpation for detection of breast cancer. U.S Pat. No. 5,833,633 issued to Sarvazyan discloses the device comprising a pressure sensor array, data acquisition circuit, and a microprocessor mounted in a hand-held pad. Detection of nodules is achieved by analyzing the dynamic and spatial features of the pressure pattern while the probe is pressed to the breast and is periodically moved transversely to the ribs. When the device detects the presence of lumps in a breast it provides a warning signal. U.S Pat. Nos. 5,916,180 and 5,989,199 issued to Cundari et al. describe several devices designated to assist the user in performing breast self-examination. These devices include an array of pressure sensors, electronic circuit and warning indicator. A plurality of processing tests is performed on the received signals from the pressure sensors, and different types of tissue structures are discriminated from each other based on the results of the tests.

All above-described devices have certain limitations. Specifically, these devices cannot be used for a regular home use in a repeatable pattern that allows for accurate and reproducible serial examinations. It is therefore desirable to provide a hand-held self-palpation device adapted for home use, which is easy to use and would facilitate regular self-examinations conducted by women, thereby leading to improvement in early detection of breast cancer.

### SUMMARY OF THE INVENTION

Accordingly, it is an object of the present invention to overcome these and other drawbacks of the prior art by providing a novel tactile image probe device capable of detecting spatial and temporal differences in tissue density via an array of sensors.

It is another object of the present invention to provide a tactile imager with automatic recordation of spatial coordinates.

It is a further object of the present invention to provide a tactile breast imager adapted for home use. More specifically, the object of the invention is to provide for greater ease of use of the imager by having a probe equipped with a positioning system to automatically transmit its positioning data to determine the probe location.

It is yet a further object of the present invention to provide a method for determining the location of a lesion in a soft tissue adapted to periodic use at home by patients without the need to involve skilled medical personnel.

The self-palpation device of the present invention utilizes the same mechanical information as obtained by manual palpation conducted by a skilled physician but does so objectively and with higher sensitivity and accuracy.

A method of detecting and locating a lesion in soft tissue is based on analyzing a sequence of pressure patterns acquired by a tactile imager probe as it is pressed against and moved over the examined tissue. The method includes the steps of evaluating position of the lesion relative to the tactile imager probe from the temporal and spatial changes of the acquired pressure patterns, estimating position of the tactile imager probe relative to an known anatomical landmark of the examined patient, and calculating position of the lesion relative to said anatomical landmark. In one embodiment of the method, a patient foreordains the scanning area and information about the scanning area location is hand entered by the patient into the hand-held self-palpation device. In another embodiment of the method, the scanning area location data is automatically detected by means of the inertial positioning system incorporated into a hand-held self-palpation device.

The nature of the invention will be more clearly understood by referencing to the following detailed description of the invention, the appended claims and the several views illustrated in the drawings.

### BRIEF DESCRIPTION OF THE DRAWINGS

A more complete appreciation of the subject matter of the present invention and the various advantages thereof can be realized by reference to the following detailed description in which reference is made to the accompanying drawings in which:

FIG. 1A is a perspective view of a first embodiment of the hand-held tactile imaging device of the present invention;

FIG. 1B is a cross-section of the device shown in FIG. 1A;

FIG. 1C is another cross-section of the device shown in FIG. 1A;

FIG. 2A is an example of raw motion tracking data of a tactile imager trajectory during a breast examination;

FIG. 2B is an example of processed trajectory first shown in FIG. 2A indicating scanned area centers relative to a landmark;

FIG. 2C illustrates reconstructed sequence of examined areas of breast calculated from the data of FIG. 2B;

FIG. 3 is an example of a sequence of 2-D tactile images of a lesion;

FIG. 4A is a perspective view of a second embodiment of the hand-held tactile imaging device;

FIG. 4B is another perspective view of the hand-held tactile imaging device shown in FIG. 4A;

FIG. 5 is a perspective view of a third embodiment of the hand-held tactile imaging device;

FIG. 6 is a perspective view of a fourth embodiment of the hand-held tactile imaging device;

FIG. 7 is a schematic diagram of an electronic circuitry for tactile sensor array; and finally

FIG. 8 is a block diagram of a hand-held tactile imaging device shown first in FIG. 1A.

### DETAILED DESCRIPTION OF THE PREFERRED EMBODIMENTS OF THE INVENTION

A detailed description of the present invention follows with reference to accompanying drawings in which like elements are indicated by like reference letters and numerals.

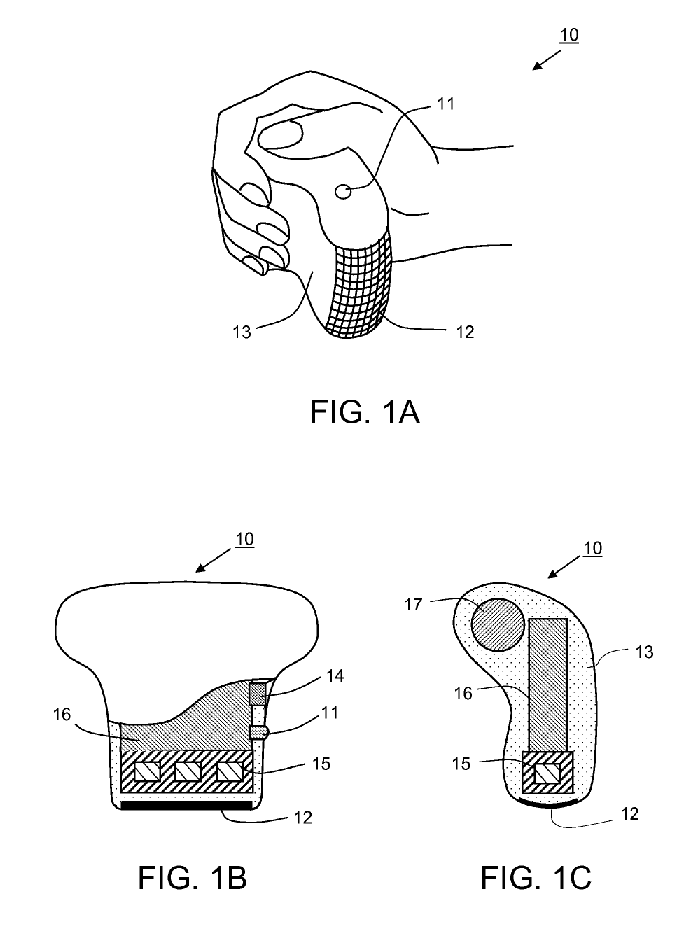

FIGS. 1A, 1B and 1C show a hand-held self-palpation tactile imaging device 10 for detecting and locating a lesion in breast tissue in accordance with a first embodiment of the present invention. The device 10 comprises pressure sensing means such as a 2-D tactile sensor array 12, a positioning data means such as an inertial motion tracking sensor 15, an electronic unit 16, a power supply 17, a computer connector 14, and an output signal source 11, all of which are mounted in a housing 13 adapted for easy grip by a human hand. Tactile sensor array 12 generates signals in response to pressure imposed on a pressure-sensing surface as it is pressed against and moved over the breast or another type of soft tissue. Tactile sensor array 12 comprises a matrix of tactile sensing elements measuring discretely contact pressure between the surfaces of the sensing elements and breast tissue. Motion tracking system 15 for generating signals in response to motion of the device 10, can include accelerometers, magnetometers, gyroscopes or a combination thereof designed to indicate the spatial coordinates of the tactile imager. The output signal source 11 generates sound or light signals when a predetermined level of total pressure on breast tissue sufficient for obtaining reliable tactile information is reached and a different type of signal when analysis of pressure patterns reveals a suspicious structure in underlying soft tissue.

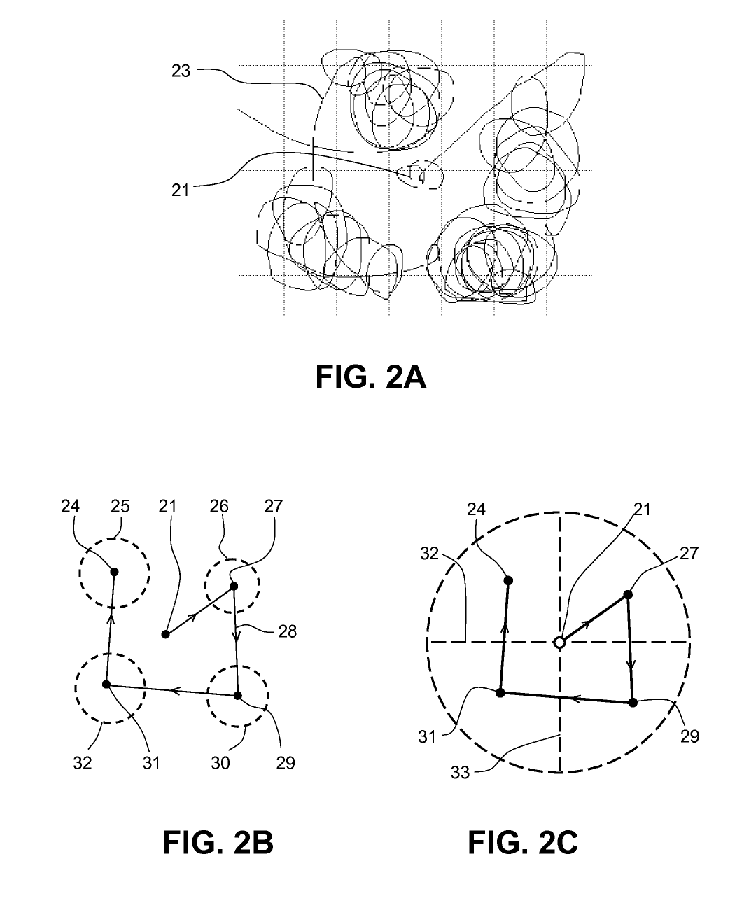

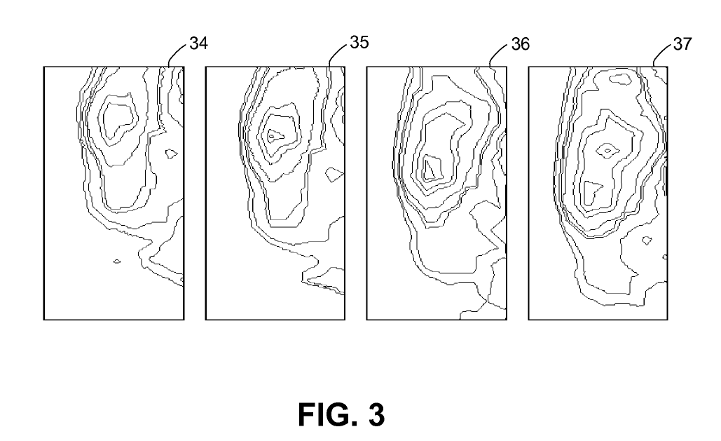

FIGS. 2A, 2B, 2C, and 3 illustrate a method of detecting and locating a lesion in breast tissue using a device shown in FIGS. 1A, 1B and 1C in accordance with the present invention. The breast self-examination procedure includes a plurality of local scans, such as for example, a sequence of four scans, one per each quadrant of examined breast. During breast self-examination, in accordance with the present invention, a tactile breast imager is moved from a starting reference point 21, which is in this case the nipple of the breast, to the quadrant of the breast to be scanned. Importantly, the starting reference point should be a known well-recognized anatomical mark or another small area on the skin, which is easy to recognize and return to at a later time, such as a nipple or a sternum. It is critical to start each subsequent examination at this same known starting reference point in order to compare the results of more than one test.

From the starting point, the imager probe is moved in a desired direction and oscillated about the desired first and subsequent areas of interest, all without letting up the minimally required pressure.

FIG. 2A is an example of a 2-D projection of the motion tracking data of the tactile imager trajectory 23 during breast self-examination. Each local scanning comprises is a result of the following steps: A. pressing the pressure sensing surface of the tactile breast imager probe against the breast tissue until a predetermined minimum level of pressure on tactile sensor array is reached as indicated by the output signal 11; and B. oscillating the pressure sensing surface about the breast tissue.

Positioning data is collected either manually by entering it by hand into a computer or into the probe itself, provided that appropriate provisions are made to the design. Preferably though, positioning a temporal data is collected automatically by using the device as described below.

After the examination is complete, the motion tracking trajectory 23 is analyzed and corrected to separate relative positions of examined breast sites 25, 26, 30, 32 by calculating the position of higher density of the trajectory lines 23 and coordinates of centers 24, 27, 29, 31 for these zones with higher density of the trajectory lines (see FIG. 2B). Then the 2-D projection of integrated trajectory 28 is mapped on a contour breast diagram with lines 32 and 33 dividing the breast into four quadrants. The starting reference point 21 corresponds to a nipple or another well-recognized point, which serves as an anatomical landmark. Concurrently, a total sequence of pressure patterns acquired by a tactile imager probe during self-examination is subdivided into a number of separate sequences of pressure patterns related to different local scanning zones. An example of pressure patters (34-37) from a dynamic image sequence for a breast local scanning is shown in FIG. 3. Temporal and spatial analysis of all pressure patterns within one zone allows reconstructing 3-D tactile image of underlying tissue structures in a relative location to the starting reference point. Then each 3-D tactile image is analyzed by artificial neural network or analytic classifiers to find features that are characteristic for the presence of a lesion or to detect changes in the breast inner mechanical structure in comparison with previously recorded (e.g. a month ago) 3-D tactile image of the same breast site.

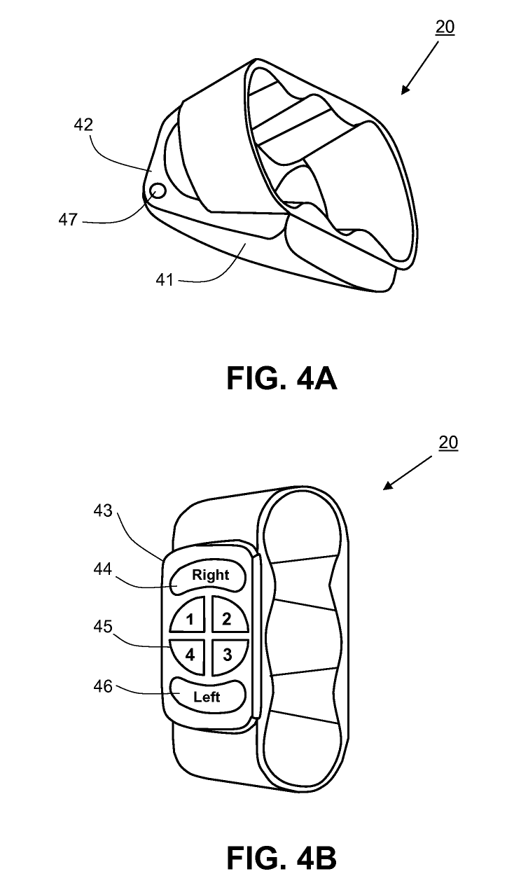

FIGS. 4A and 4B show a hand-held self-palpation device 20 for detecting and locating a lesion in breast tissue in accordance with a second embodiment of the present invention. The device 20 comprises 2-D tactile sensor array 41, a patchboard 43 with buttons 44-46, an output signal source 47, all of which are mounted in a housing 42. During the breast self-examination, a patient is putting this device on her fingertips. Tactile sensor array 41 generates signals in response to pressure imposed on a pressure-sensing surface as it is pressed against and moved over the breast. The buttons 44, 45, 46 are intended for hand entering information about which breast and which quadrant of the breast is under examination. The patchboard 43 and housing 42 include all necessary electronics for storing and preliminary processing acquired tactile data. Scanning area location data are stored in a device recording system. After the breast examination is complete, the examination data can be transmitted to a home computer.

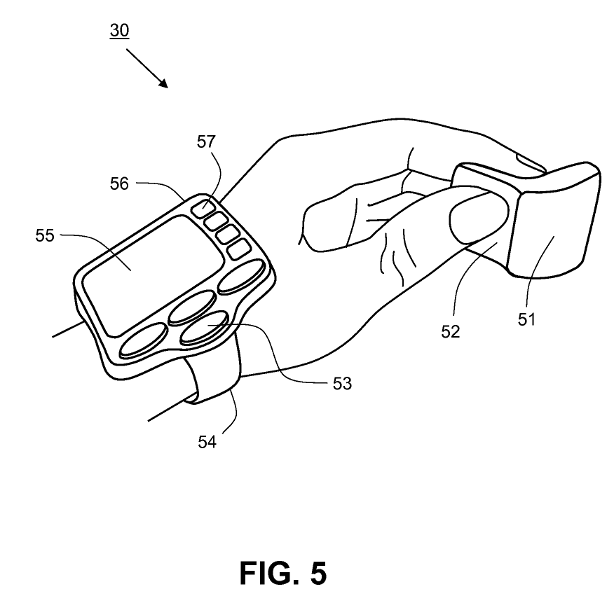

FIG. 5 represents a hand-held self-palpation device 30 for detecting and locating a lesion in breast tissue in accordance with a third embodiment of the present invention. The device 30 comprises 2-D tactile sensor array 51 mounted on a touch pad 52 and an electronic unit 56 with a display means 55 optionally secured on a patient's wrist or hand by a strap 54. During the breast self-examination, the patient can in real-time environment observe the results of breast examination and communicate with device 30 by means of control buttons 53. Along with that the patient can enter the information about position of the local scanning zone using data entry means such as buttons 57. The device 30 allows transmission of received breast examination data into a home computer.

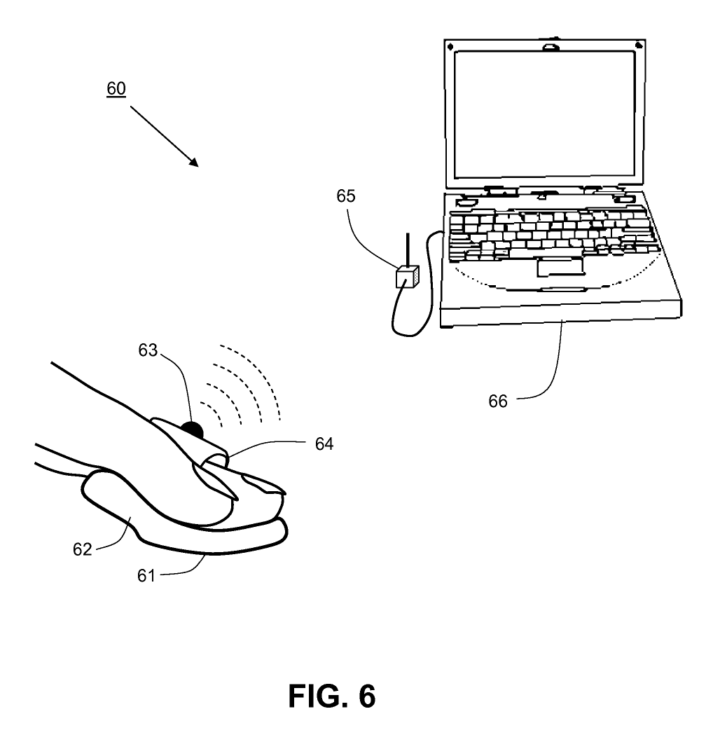

FIG. 6 represents a hand-held self-palpation device 60 for detecting and locating a lesion in breast tissue in accordance with a fourth embodiment of the present invention. The device 60 comprises a touch pad 62 with 2-D tactile sensor array 61 and electronics required for signal acquisition from the tactile sensor and follow-up data transmission through wireless transmitter 63 to a receiver 65 connected in turn with a home computer 66. The touch pad 62 is distinct in a sense that the pressure sensing surface of tactile sensor array 62 is located in close proximity to patient's fingertips and touch pad 62 can be secured on patient's finger(s) by a finger strap 64. The patient can inspect breast self-examination results on a computer screen both during breast self-examination in real-time and after the breast examination is complete. The patient can also enter into computer the information about position of the local scanning zone for subsequent data storing and analysis.

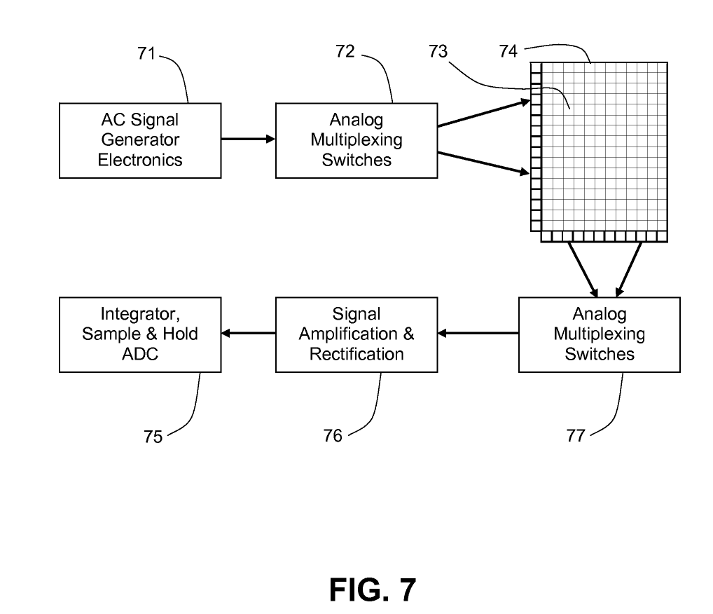

Tactile sensor arrays used in tactile breast imager can be based on different types of sensors, such as resistive, capacitive, piezoelectric, or fiber optic. FIG. 7 shows a block diagram of a capacitive pressure sensor array 74 and a corresponding analog measurement system that can be used in the tactile imager of the present invention. The sensor element/pixel is formed by orthogonal intersection of two current-conducting strips separated by a thin air gap partly filled by an elastic dielectric substrate. Force applied above the sensing element causes the strips to draw closer together, thus increasing the capacitance. By measuring the change in capacitance of each sensor element in the array, the local pressure above each element can be determined. The geometry of the sensor sets the base capacitance and the relative increase in capacitance due to the applied pressure. Typically, the sensors base capacitance is in a range from about 10 to about 100 pF and the relative change is on the order of about 5% to about 20%. Before a measurement is started, the central processing unit (CPU) control system sets up the analog multiplexers 72 and 77 to select a particular element in the array to be measured as well as configures the offset and gain correction factors for that pixel. Once these parameters are set, AC signal generator 71 generates a sine wave with the frequency between about 50 kHz to about 200 kHz. The frequency can be varied depending on the sensor size and the array scanning rate. The output signal from the sensor element is compared with a signal from a reference capacitor and after amplifying/rectifying in block 76, a net signal is sent to the integrator 75. After integrator 75 there is placed an analog-to-digital converter, which captures the final output signal proportionally to the force applied above the sensing element. Utilizing a cross-multiplexing technique allows minimizing electrical and mechanical components in the tactile pressure array.

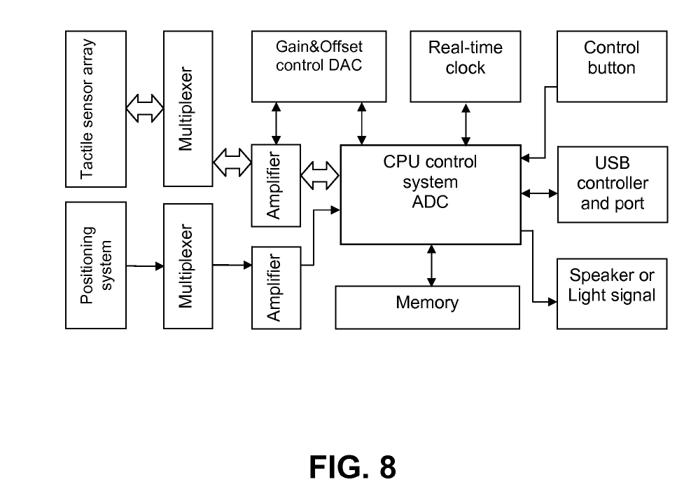

FIG. 8 shows a block diagram of tactile breast imager, such as that shown in FIGS. 1A, 1B, and 1C, comprising a tactile sensor array, positioning system, multiplexers and signal conditioning electronics, CPU control system with digital-to-analog converter, memory block, computer controller, controls and indicators. After the device is turned on, the initialization parameters are loaded into the memory, a hardware testing procedure is performed, and an acquisition process is initiated. As the first data is accumulated, the null values for the sensor elements and the noise level are estimated. The device then turns to the “stand by” mode in which the signals from the array are acquired at a very slow rate in the range of 1-5 Hz. After the tactile imager probe is pressed against the breast and signals from most of the tactile sensors reach a certain predetermined value, the examination mode is initiated. The acquisition frequency rises up to the frequency in the range of about 30 to about 100 Hz. The probe is pressed against the breast at the chosen quadrant or local scanning zone and periodically oscillated in a linear or circular manner parallel to the plane of examined breast tissue. The primary raw data obtained during examination is saved into the memory buffer, as frames of the 2-D pressure patterns. After the data pass through the refinement block where the filtration and noise reduction are carried out, the quality of received data is estimated, and after that total sequence of the pressure patterns is analyzed in accordance with the above described method. Switching off the device initiates the closing procedure. The null values as noise level are checked again, the acquired information is packed and the program shuts down. The real time clock provides for maintaining a desired data acquisition frequency and relates the data to the examination time scale.

Although the invention herein has been described with respect to particular embodiments, it is understood that these embodiments are merely illustrative of the principles and applications of the present invention. For example, any soft tissue may be examined with the help of the device of the invention in addition to breast tissue. It is therefore to be understood that numerous modifications may be made to the illustrative embodiments and that other arrangements may be devised without departing from the spirit and scope of the present invention as defined by the appended claims.

What is claimed is:

1. A hand-held self-palpation tactile imager probe for detecting and locating a lesion in soft tissue, said probe comprising:
a housing adapted to fit over fingers of a human hand, said housing having a lower portion and an upper portion,
a pressure sensing means located on said lower portion of said housing and including a 2-D pressure sensor array facing towards said soft tissue,
a patchboard incorporated with said upper portion of said housing, said patchboard adapted for manual entry of positioning data of said probe over said soft tissue, and
an output signal source.

2. The probe as in claim 1, wherein said soft tissue is human breast, said patchboard further including a plurality of buttons adapted to manually enter the positioning data of said probe in such a way as to discriminate between left and right breast, said plurality of buttons further adapted to discriminate the position of said probe over the breast in a respective upper-, lower-, right-, or left quadrants.

3. The probe as in claim 2, wherein said patchboard is equipped with a “left” and “right” button so as to allow manual entry of positioning data of said device over a respective left or right breast.

4. The probe as in claim 4 further including a four-segmented button to indicate the positioning data of said probe over a respective quadrant of said breast, said quadrant being an upper-, a lower-, a right- or a left quadrant.

5. A self-palpation tactile imager probe for detecting and locating a lesion in soft tissue, said probe comprising:
a pressure sensing means adapted to fit in a human hand and including a 2-D pressure sensor array facing towards said soft tissue,
a hand-mounted housing having an upper portion,
a patchboard incorporated with said upper portion of said housing, said patchboard adapted for manual entry of positioning data of said sensing means over said soft tissue, and
an output signal source.

6. The probe as in claim 5 further including a display means for real-time observation of self-palpation examination results.

7. The probe as in claim 5, wherein said soft tissue is human breast, said patchboard further including a plurality of buttons adapted to manually enter the positioning data of said probe in such a way as to discriminate between left and right breast, said plurality of buttons further adapted to discriminate the position of said probe over the breast in a respective upper-, lower-, right-, or left quadrants.

8. The probe as in claim 7, wherein said patchboard is equipped with a “left” and “right” button so as to allow manual entry of positioning data of said device over a respective left or right breast.

9. The probe as in claim 8 further including four buttons to indicate the positioning data of said probe over a respective quadrant of said breast, said quadrant being an upper-, a lower-, a right- or a left quadrant.

10. The probe as in claim 5, wherein said output signal source further including transmission means to a remote network.

11. The probe as in claim 10, wherein said transmission means are wireless.

12. A self-palpation tactile imager probe for detecting and locating a lesion in soft tissue, said probe comprising:
a pressure sensing means adapted to fit in close proximity to human fingers and including a 2-D pressure sensor array facing towards said soft tissue,
a wireless transmission means to a personal computer,
a means to manually enter the positioning data of said probe over said soft tissue in said personal computer.

13. The probe as in claim 12, wherein said pressure sensing means is equipped with a finger strap.

14. The probe as in claim 12, wherein said soft tissue is human breast, said means to manually enter the positioning data in the personal computer include means to discriminate between left and right breast as well as the appropriate quadrant over which the probe is located, said quadrant including upper-, lower-, right-, or left quadrant of the breast.

15. The probe as in claim 12, wherein said pressure sensing means is a capacitive tactile pressure sensor array.

16. The probe as in claim 15, wherein each sensor of said sensor array is formed by intersection of two current-conducting strips separated by a gap, said gap at least partially filled with an elastic dielectric substrate.

17. The probe as in claim 12 further including a multiplexer adapted to select an element of said array to be measured and a central processing unit to control data acquisition, processing and transmission.

18. The probe as in claim 17, wherein said multiplexer provides for cross-multiplexing all of said sensors in said sensor array to a signal detection circuit, whereby the overall size of the probe is minimized.
